(19) United States
(12) Patent Application Publication
(10) Pub. No.: US 2012/0107811 A1
(43) Pub. Date: May 3, 2012

(54) BURSTABLE LIQUID PACKAGING AND USES THEREOF

(76) Inventors: David M. Kelso, Wilmette, IL (US); Abhishek Agarwal, Evanston, IL (US); Kunai Sur, Evanston, IL (US)

(21) Appl. No.: 13/148,036

(22) PCT Filed: Feb. 5, 2010
(86) PCT No.: PCT/US10/23310

§ 371 Date: Jan. 23, 2012

Related U.S. Application Data
(63) Continuation of application No. 61/150,481, filed on Feb. 6, 2009.

(51) Int. Cl. C12Q 1/68; C12M 1/34

(52) U.S. Cl. 435/6.11; 435/287.2

(57) ABSTRACT

The present invention relates to systems, devices, and methods for performing biological and chemical reactions. In particular, the present invention relates to the use of burstable liquid packaging for delivery of reagents to biological and chemical assays.

### CROSS REFERENCE TO RELATED APPLICATIONS

This application claims priority to provisional application 61/150,481, filed Feb. 6, 2009, which is herein incorporated by reference in its entirety.

### FIELD OF THE INVENTION

The present invention relates to systems, devices, and methods for performing biological and chemical reactions. In particular, the present invention relates to the use of burstable liquid packaging for delivery of reagents to biological and chemical assays.

### BACKGROUND OF THE INVENTION

Many existing methods of storing liquid reagents used in medical diagnostics are done in sterilized plastic bottles that often require cold chain technology for shipping, transportation and storage at the final destination. Although such methods are feasible in most developed nations, such a requirement poses challenges and presents higher costs for developing nations. There may be issues during shipping, customs, and provision of reliable and consistent electricity for the refrigeration equipment at the site for storing the reagents. Any one of these has the potential to expose the reagents to high temperatures, rendering them useless for clinical use. Furthermore, because the reagents are stored and delivered in bulk, a skilled clinical laboratory technician and precision fluid-handling equipment are often required for precision pipetting and aliquoting for the individual medical diagnostic tests. This manual operation increase cross-contamination between samples, takes additional processing time, and increases the cost of administering and processing a diagnostic test.

Depending on how a diagnostic system operates, liquid delivery to a diagnostic test cartridge can be done using precision pipetting, or directly through the stock liquid reagent bottles via tubing, precision pumps, and valves. Such fluidic components add increased cost and complexity to the design of the diagnostic system. Furthermore, they are often prone to contamination, failure (requiring mechanical servicing and/or replacement), and leaks. Additional methods of storing and delivery reagents are needed. In particular need are compositions and methods for transporting and storing reagents at ambient temperature.

### SUMMARY OF THE INVENTION

The present invention relates to systems, devices, and methods for performing biological and chemical reactions. In particular, the present invention relates to the use of burstable liquid packaging for delivery of reagents to biological and chemical assays. In some embodiments, the present invention provides assay systems and methods of their use, comprising: a liquid packaging component comprising one or more liquid storage compartments, wherein the liquid storage compartments comprise a liquid and are covered with a burstable seal; a seal bursting component configured to burst the burstable seal; and an assay device configured to accept liquids from the liquid storage compartments. In some embodiments, the burstable seal is a foil (e.g., aluminum) laminate. In some embodiments, the laminate comprises aluminum foil sandwiched between a protective plastic film and a heat-sensitive sealant. In some embodiments, the seals are peelable or permanent. In some embodiments, the seal bursting component comprises plungers that compress said liquid storage compartment under conditions such that the seals are burst (e.g., by peeling open). In some embodiments, the plungers are driven by one or more motors. In other embodiments, the plungers are manually driven. In some embodiments, the liquid packaging component and chamber within the assay device are connected via fluid conduits or are in direct contact. In some embodiments, the liquid packaging component comprises one or more liquid storage compartments. In some embodiments, the liquid storage compartments comprise less than 60%, and preferably less than 50% air by volume. In some embodiments, the liquid storage compartments comprise less than 400 μl air. In some embodiments, the liquid packaging component further comprises one or more alignment pins to secure the liquid storage compartment to the liquid packaging component.

In some embodiments, the liquid storage compartment comprises a tear-drop clamp that applies uniform pressure across a portion of the burstable seal perimeter. In some embodiments, the tear drop clamp does not apply pressure to the portion of the burstable seal that is in communication with the assay device. In some embodiments, the liquid storage compartments comprise reagents for performing a biological or chemical assay. In some embodiments, the assay is selected from a diagnostic assay or a research assay (e.g., nucleic acid based assays (e.g., PCR) or protein based assays).

### DESCRIPTION OF THE FIGURES

FIG. 1. Cross-sectional diagram of a typical (opaque) high vapor and oxygen barrier aluminum (Al) foil laminate. (b) a thin sheet of Al foil that behaves as the barrier, and (c) a thin protective plastic film that prevents the Al foil from being damaged or torn during handling and processing.

FIG. 2. Process diagram showing how a blister (here, hemispherical) is made in a high vapor and oxygen barrier laminate that can be pressure formed. (a) The cold forming station comprises a male plug with vent hole, stripper plate with a through-hole to allow the male plug to pass through, and a matching female cavity with vent hole. A corner radius is machined into the female cavity to prevent the laminate film from tearing/pinching during cold-forming. (b) Pressure is applied on the stripper plate to hold the laminate film firmly. Next, pressure is applied on the male plug to create the blister shape. (c) Liquid can be precisely aliquoted into the cold-formed blister (top); a photograph of a blister is also shown (bottom).

FIG. 3. Diagram of a cold-formed high vapor, oxygen, and UV barrier (Al foil) laminate blister with liquid. The apex of the liquid droplet is in the same plane as the top of the laminate blister. The cross-section of a typical Al foil laminate is show on the right. The liquid rests on the heat-sensitive sealant side of the blister laminate.

FIG. 4. Two types of heat seals—peelable and permanent. Peelable seals (top) are fabricated at lower temperatures using different sealant materials. They have a lower peel strength and are designed to be opened post-manufacturing. Permanent seals (middle) are typically fabricated at higher temperatures using similar sealant materials and have higher peel strengths. The heat sealing can also be extended to bonding laminate to rigid plastics, as shown in the bottom image.

FIG. 5. Design schematics of the contour (impulse) heat seal press (left) used to seal the liquids inside the cold-formed blister. The upper platen (top right) holds an interchangeable heat seal (here, a circular geometry) band that is designed to match a blister's geometry. The lower platen (bottom right) holds an interchangeable silicone/aluminum pressure also designed to match the blister geometry.

FIG. 6. General process for heat sealing a cold-formed Al foil laminate blister to store liquids. (a-b) The cold formed-blister is positioned in the lower platen relief. Foil laminate #2 is positioned on top such that the sealants face one another. (c) The upper platen, with the heat seal band, comes down on top of the blister, applying synchronized pressure and heat to heat seal (peelable) the two laminates together. (d) The final packaged and heat sealed blister.

FIG. 7. Cross-sectional diagrams of liquid stored in a heat sealed (peelable) Al foil laminate blister. (a) Parameters of the heat sealing process are: x—distance between the edge of the blister and heat seal band; w—heat seal width. (b) Due to the Al foil laminate in both foil laminates, liquid loss does not occur through the film, but only through the heat seals. It is a function of the blister volume, ambient temperature, heat-sensitive sealant material properties (permeability), heat seal surface area (a function of w), and tseal (thickness of the final heat seal).

FIG. 8. Perspective and cross-sectional diagrams showing how a packaged blister may be integrated with a rigid, disposable (plastic) cartridge using double-sided tape. (a) Conceptual schematics showing a perspective view (solid view—left; transparent view—right) of the cartridge and integrated blister. (b) Double-sided adhesive is bonded to the cartridge, with appropriate slits for the input port(s). The packaged blister is subsequently bonded to the opposite side of the double-sided adhesive. Foil laminate #2 is extended beyond the blister and has a matching slit that is aligned with the input port. (c) Alternative method for (b) where the cold-formed laminate is not adhesively bonded to the cartridge. (d-e) Alternative methods—foil laminate #2 is trimmed to the blister size and the packaged blister is bonded to the cartridge using double-sided.

FIG. 9. Cross-sectional diagrams showing how a packaged blister may be integrated with a rigid, disposable (plastic) cartridge using heat seals, with the option of adding double-sided adhesive. (a) The packaged blister is heat sealed to the rigid cartridge. The heat-sensitive sealant on the cold-formed blister laminate is similar to the rigid cartridge material. (b) Alternative method—Foil laminate #2 is adhesively bonded to the rigid cartridge. The cold-formed blister laminate is subsequently heat sealed to the rigid cartridge.

FIG. 10. Mechanical clamping mechanism to burst a packaged blister to deliver the liquid to the cartridge chamber. The initial state of this example cartridge is shown in FIG. 8(e). (a-b) Cross-sectional (left) and corresponding top-view (right) diagrams. A mechanical clamp is positioned around the edge of the blister and around the input port of the rigid cartridge to ensure the liquid from the blister will only flow in one direction—from the blister to the input port. A mechanical plunger is used to apply uniform pressure on the blister until the peelable seal breaks, allowing the stored liquid to escape, enter the input port, and flow into the cartridge chamber.

FIG. 11. Schematic showing how an exemplary rigid cartridge with three packaged blisters may interface with the mechanical clamping and bursting module. The current design shows three separate linear motors operating each of the mechanical clamp and plunger combination, but has the potential for being driven by a single linear stepper motor.

FIG. 12. Cross-sectional diagrams showing an integrated cartridge and packaged blister with both peelable and permanent heat seals. (a) Initial state of the cartridge—same configuration shown in FIG. 9(b). (b) A mechanical plunger is aligned with and pressed against the cold-formed blister. Due to its lower peel strength, the peelable seal breaks in a random location, and the liquid flows out of the blister into the cartridge chamber. The permanent heat seal does not burst.

FIG. 13. An example of a diagnostic cartridge that has three packaged foil laminate blisters integrated within it on the underside which store three separate aqueous and/or non-aqueous liquids. The diagrams also illustrate how a lyophilized assay bead may be readily integrated into the cartridge for dissolution with the respective liquid. A hydrophobic air permeable membrane provides a point for air to escape as liquid is dispensed into the respective chambers. (a) The initial state of the cartridge. (b) A blister is burst open, dispensing the liquid through the input port and channel into chamber 3. The lyophilized bead dissolves. (c) The second blister is burst open, dispensing the second liquid in a similar fashion. (d) The third blister is burst open similarly. Here, this blister has a non-aqueous liquid which helps prevent contamination by separating chamber 3 from both chamber 1 and overflow.

FIG. 14. Photograph of a blister crusher. (a) Front view of a 3-blister crusher showing the three tear-drop clamps and plungers that mate with the respective blisters. (b) Side view of the 3-blister crusher showing the tear-drop clamps and springs that provide the clamping force. Here, individual stepper motors drive each of the plungers. The cartridge fits between the aluminum plate and mounting plate. (c) Schematic of the cartridge showing the position of the three respective blisters (shown by the concentric gray circles), input ports, channels, and chambers.

FIG. 15. Exemplary blister crushing mechanism. (a) A cross-sectional diagram of a fluidic cartridge that has an adhesively bonded blister on the backside. The blister is positioned such that the top of the heat seal perimeter is directly below the input port. (b) A stepper motor is used to apply pressure on most of the heat seal perimeter via the tear-drop clamp. Next, a plunger, also driven by the stepper motor, presses against the blister and bursts the peelable heat seal nearest to the input port. Air escapes into the channel and chambers, followed by the liquid reagent. (c) Top view diagram showing a blister and the position of the tear-drop clamp. The additional clamping force prevents the heat seals in the clamped areas from being burst open. Only at the location marked by x does the heat seal peel open, allowing first the air to escape, and subsequently, the liquid reagent.

FIG. 16. Example schematic of a modified fluidic cartridge (front and back views) showing three blisters and how they are interfaced with the fluidic cartridge, and additional plastic pieces (blister guards) to ensure the blisters only burst in one specified location—marked by the x.

FIG. 17. (a) Photograph of one of the three tear-drop clamps showing the plunger (with relief) inside. An o-ring on the outside periphery of the tear-drop clamp ensures intimate contact with the blister surface. (b) Photograph of a cartridge positioned in the blister crusher. The inset photo shows the position of the blister with respect to the input port on the fluidic cartridge.

FIG. 18. Graph showing the forces required to crush the respective blisters—elution, oil, and lysis. The graph shows that both the size of the blister, and liquid volume seem to affect the force required to burst open the blisters. Vertical bars show the standard deviation.

FIG. 19. Data plot showing the effect of blister stroke on the liquid volume fill capacity for two different blister diameters—0.55″ and 0.72″.

FIG. 20. Schematic of a characterization cartridge used to determine the dead volume for a given blister geometry and fill volume.

FIG. 21. Data plot showing the effect of liquid volume in given blister geometry on the load (force) required to burst it open.

FIG. 22. Diagram of a cold formed blister.

FIG. 23. (a) Top-view schematic of a cold-formed blister in a foil laminate.

Two alignment holes are punched through the foil laminate that serve as alignment guides. They are punched along the central axis (dashed line) point of the cold-formed blister and are outside the circular heat seal width (dashed-dotted line). (b) Trimetric schematic view of the cold-formed blister in a foil laminate showing the blister and two alignment holes.

FIG. 24. Top-view schematic of the lidstock. Three holes are punched into the lidstock material—two serve as alignment holes for the heat sealing process, and subsequent integration and positioning with the rigid test cartridge; the third hole serves as the liquid thru port.

FIG. 25. Brief outline of the heat sealing procedure, which shows how the cold-formed blister (a) is aligned with the lidstock (b). Retractable alignment pins on the lower platen of the impulse heat sealer facilitate the positioning and alignment (c-f).

FIG. 26. (a) Schematic of the general heat seal band used for heat sealing the blisters. It has an active area in the geometry of a donut ring with tab extensions on two sides, which overlap with the three punched holes on the cold-formed blister and lidstock.

The inactive area is for electrical contact and does not promote heat sealing. (b) Top-view of a heat sealed blister showing the heat seal perimeter (outlined by the dashed-dotted line) and how it overlaps the three punched holes. (c) A heat sealed blister which has been punch-cut to the desired profile shape (i.e., trimmed around the heat seal perimeter).

FIG. 27. (Left) Cross-sectional views of the rigid assay cartridge with a double-sided transfer adhesive bonded to it. A packaged blister with the three punched holes can be bonded to the rigid assay cartridge (shown on the right). The bonding is on the same level across the blister and therefore, there are no channels created as before.

FIG. 28. (a) Top-view schematic of a cold-formed blister with reagent. (b-c) Two embodiments showing mechanical clamps positioned on the respective blister, aligned using the alignment holes. Pressure from the clamp is applied on the heat seal perimeter of the blister.

### DEFINITIONS

To facilitate an understanding of this disclosure, terms are defined below:

“Purified polypeptide” or “purified protein” or “purified nucleic acid” means a polypeptide or nucleic acid of interest or fragment thereof which is essentially free of, e.g., contains less than about 50%, preferably less than about 70%, and more preferably less than about 90%, cellular components with which the polypeptide or polynucleotide of interest is naturally associated.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

“Polypeptide” and “protein” are used interchangeably herein and include all polypeptides as described below. The basic structure of polypeptides is well known and has been described in innumerable textbooks and other publications in the art. In this context, the term is used herein to refer to any peptide or protein comprising two or more amino acids joined to each other in a linear chain by peptide bonds. As used herein, the term refers to both short chains, which also commonly are referred to in the art as peptides, oligopeptides and oligomers, for example, and to longer chains, which generally are referred to in the art as proteins, of which there are many types.

A “fragment” of a specified polypeptide refers to an amino acid sequence which comprises at least about 3-5 amino acids, more preferably at least about 8-10 amino acids, and even more preferably at least about 15-20 amino acids derived from the specified polypeptide.

The term “immunologically identifiable with/as” refers to the presence of epitope(s) and polypeptide(s) which also are present in and are unique to the designated polypeptide(s). Immunological identity may be determined by antibody binding and/or competition in binding. The uniqueness of an epitope also can be determined by computer searches of known data banks, such as GenBank, for the polynucleotide sequence which encodes the epitope and by amino acid sequence comparisons with other known proteins.

As used herein, “epitope” means an antigenic determinant of a polypeptide or protein. Conceivably, an epitope can comprise three amino acids in a spatial conformation which is unique to the epitope. Generally, an epitope consists of at least five such amino acids and more usually, it consists of at least eight to ten amino acids. Methods of examining spatial conformation are known in the art and include, for example, x-ray crystallography and two-dimensional nuclear magnetic resonance.

A “conformational epitope” is an epitope that is comprised of a specific juxtaposition of amino acids in an immunologically recognizable structure, such amino acids being present on the same polypeptide in a contiguous or non-contiguous order or present on different polypeptides.

A polypeptide is “immunologically reactive” with an antibody when it binds to an antibody due to antibody recognition of a specific epitope contained within the polypeptide. Immunological reactivity may be determined by antibody binding, more particularly, by the kinetics of antibody binding, and/or by competition in binding using as competitor(s) a known polypeptide(s) containing an epitope against which the antibody is directed. The methods for determining whether a polypeptide is immunologically reactive with an antibody are known in the art.

As used herein, the term “immunogenic polypeptide containing an epitope of interest” means naturally occurring polypeptides of interest or fragments thereof, as well as polypeptides prepared by other means, for example, by chemical synthesis or the expression of the polypeptide in a recombinant organism.

“Purified product” refers to a preparation of the product which has been isolated from the cellular constituents with which the product is normally associated and from other types of cells which may be present in the sample of interest.

“Analyte,” as used herein, is the substance to be detected which may be present in the test sample, including, biological molecules of interest, small molecules, pathogens, and the like. The analyte can include a protein, a polypeptide, an amino acid, a nucleotide target and the like. The analyte can be soluble in a body fluid such as blood, blood plasma or serum, urine or the like. The analyte can be in a tissue, either on a cell surface or within a cell. The analyte can be on or in a cell dispersed in a body fluid such as blood, urine, breast aspirate, or obtained as a biopsy sample.

A “capture reagent,” as used herein, refers to an unlabeled specific binding member which is specific either for the analyte as in a sandwich assay, for the indicator reagent or analyte as in a competitive assay, or for an ancillary specific binding member, which itself is specific for the analyte, as in an indirect assay. The capture reagent can be directly or indirectly bound to a solid phase material before the performance of the assay or during the performance of the assay, thereby enabling the separation of immobilized complexes from the test sample.

The “indicator reagent” comprises a “signal-generating compound” (“label”) which is capable of generating and generates a measurable signal detectable by external means. In some embodiments, the indicator reagent is conjugated (“attached”) to a specific binding member. In addition to being an antibody member of a specific binding pair, the indicator reagent also can be a member of any specific binding pair, including either hapten-anti-hapten systems such as biotin or anti-biotin, avidin or biotin, a carbohydrate or a lectin, a complementary nucleotide sequence, an effector or a receptor molecule, an enzyme cofactor and an enzyme, an enzyme inhibitor or an enzyme and the like. An immunoreactive specific binding member can be an antibody, an antigen, or an antibody/antigen complex that is capable of binding either to the polypeptide of interest as in a sandwich assay, to the capture reagent as in a competitive assay, or to the ancillary specific binding member as in an indirect assay. When describing probes and probe assays, the term “reporter molecule” may be used. A reporter molecule comprises a signal generating compound as described hereinabove conjugated to a specific binding member of a specific binding pair, such as carbazole or adamantane.

The various “signal-generating compounds” (labels) contemplated include chromagens, catalysts such as enzymes, luminescent compounds such as fluorescein and rhodamine, chemiluminescent compounds such as dioxetanes, acridiniums, phenanthridiniums and luminol, radioactive elements and direct visual labels. Examples of enzymes include alkaline phosphatase, horseradish peroxidase, beta-galactosidase and the like. The selection of a particular label is not critical, but it should be capable of producing a signal either by itself or in conjunction with one or more additional substances.

“Solid phases” (“solid supports”) are known to those in the art and include the walls of wells of a reaction tray, test tubes, polystyrene beads, magnetic or non-magnetic beads, nitrocellulose strips, membranes, microparticles such as latex particles, and others. The “solid phase” is not critical and can be selected by one skilled in the art. Thus, latex particles, microparticles, magnetic or non-magnetic beads, membranes, plastic tubes, walls of microtiter wells, glass or silicon chips, are all suitable examples. It is contemplated and within the scope of the present invention that the solid phase also can comprise any suitable porous material.

As used herein, the terms “detect”, “detecting”, or “detection” may describe either the general act of discovering or discerning or the specific observation of a detectably labeled composition.

The term “polynucleotide” refers to a polymer of ribonucleic acid (RNA), deoxyribonucleic acid (DNA), modified RNA or DNA, or RNA or DNA mimetics. This term, therefore, includes polynucleotides composed of naturally-occurring nucleobases, sugars and covalent internucleoside (backbone) linkages as well as polynucleotides having non-naturally-occurring portions which function similarly. Such modified or substituted polynucleotides are well-known in the art and for the purposes of the present invention, are referred to as “analogues.”

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “nucleic acid amplification reagents” includes conventional reagents employed in amplification reactions and includes, but is not limited to, one or more enzymes having polymerase activity, enzyme cofactors (such as magnesium or nicotinamide adenine dinucleotide (NAD)), salts, buffers, deoxynucleotide triphosphates (dNTPs; for example, deoxyadenosine triphosphate, deoxyguanosine triphosphate, deoxycytidine triphosphate and deoxythymidine triphosphate) and other reagents that modulate the activity of the polymerase enzyme or the specificity of the primers.

As used herein, the terms “complementary” or “complementarity” are used in reference to polynucleotides (i.e., a sequence of nucleotides such as an oligonucleotide or a target nucleic acid) related by the base-pairing rules. Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids.

The term “homology” refers to a degree of identity. There may be partial homology or complete homology. A partially identical sequence is one that is less than 100% identical to another sequence.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tm of the formed hybrid, and the G:C ratio within the nucleic acids.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl (see e.g., Anderson and Young, Quantitative Filter Hybridization, in Nucleic Acid Hybridization (1985). Other references include more sophisticated computations which take structural as well as sequence characteristics into account for the calculation of Tm.

As used herein the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids which are not completely complementary to one another be hybridized or annealed together.

The term “wild-type” refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “oligonucleotide” as used herein is defined as a molecule comprised of two or more deoxyribonucleotides or ribonucleotides, preferably at least 5 nucleotides, more preferably at least about 10-15 nucleotides and more preferably at least about 15 to 30 nucleotides, or longer. The exact size will depend on many factors, which in turn depends on the ultimate function or use of the oligonucleotide. The oligonucleotide may be generated in any manner, including chemical synthesis, DNA replication, reverse transcription, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide.

The term “primer” refers to an oligonucleotide which is capable of acting as a point of initiation of synthesis when placed under conditions in which primer extension is initiated. An oligonucleotide “primer” may occur naturally, as in a purified restriction digest or may be produced synthetically.

### DETAILED DESCRIPTION OF THE INVENTION

The present invention relates to systems, devices, and methods for performing biological and chemical reactions. In particular, the present invention relates to the use of burstable liquid packaging for delivery of reagents to biological and chemical assays.

In some embodiments, the present invention provides a disposable liquid packaging module that stores liquids, both aqueous and nonaqueous, in sealed high vapor, oxygen, and UV barrier laminates (e.g., aluminum foil laminates) blisters, and has the capacity to deliver the fluids by bursting the seals using applied pressure.

In some embodiments, such packaging modules are used to dispense liquid into channels and respective fluidic chambers in an assay device such as, for example, a rigid (e.g., plastic disposable) diagnostic cartridge. In some embodiments, laminates, which have a sealant layer on one side, are cold-formed using pressure to create a hemispherical blister appropriately sized for the necessary liquid volume; liquids are precisely aliquoted into the formed blisters; a secondary flat laminate with a different sealant material is placed on top and a perimeter heat seal is made using a heat sealer (the seal may also be made, for example, using ultrasonic, radio frequency, and laser welding techniques). The packaged blister is aligned and adhered to the rigid cartridge, which contains an input port for fluid entry and connecting channel to the fluidic chamber. By application of a controlled pressure on the blister, the heat seal can be burst open, allowing the fluid to enter the input port, and flow through the channel into the respective chamber in the plastic cartridge. One example use of the diagnostic cartridge is for polymerase chain reaction (PCR) based detection and analysis for infections including, but not limited to HIV, Chlamydia, and Gonorrhea or other pathogens or analytes of interest.

This method of packaging and delivering liquids is designed and developed for any number of diagnostic and clinical uses, although it especially serves point-of-care and resource-limited settings, where refrigeration and cold chain technologies are not consistently available. It enables the medical diagnostic cartridge to be self-sufficient since the appropriate liquid reagents are packaged with the test. The high vapor, oxygen, and UV barrier laminates prevent contamination and evaporation of the small liquid volumes. The method of bursting the pouches and delivering the fluids to a secondary location removes the necessity of additional fluidic components, such as pumps, valves, and precision liquid metering units.

Embodiments of the invention disclosed herein provide many benefits to overcome challenges associated with existing technology:

Self-sufficient test cartridge with on-chip liquid reagents

High vapor, oxygen, and UV barrier storage blisters using low cost Al foil laminates prevent contamination and evaporation of the liquid until its use

Removal of complex and costly fluid handling components, such as precision pumps, valves, and tubing

Elimination of cold chain technology when integrated with lyophilized assay beads

Simple liquid delivery mechanism by applying controlled pressure on the blister and bursting its seal

Precision aliquoting of the reagents into the blisters can be done at the manufacturing site, reducing the complexity at the clinical setting

### I. Liquid Storage Component

As described above, embodiments of the present invention provide liquid storage components comprising one or more blisters with burstable seals. In some exemplary embodiments, the blisters are made with the following steps and utilized in the following exemplary applications. The invention is not limited to these exemplary embodiments. Each of the following steps is described in more detail below.

1. A high vapor, oxygen, and UV barrier laminate is cold-formed using pressure to create a hemispherical blister;

2. Liquid is precisely aliquoted into the blister (e.g., in a laboratory setting, using a manual hand-operated pipet);

3. A perimeter heat seal is created between the cold-formed laminate blister and secondary laminate using one of many available heat sealing technologies (e.g., resistive, laser, radio frequency, ultrasonic);

4. Integrating a packaged blister with a rigid plastic cartridge;

5. Bursting the blister is realized by placing a mechanical clamp around the blister's heat seal and input port in the plastic cartridge and applying uniform pressure on the hemispherical blister until the seal breaks between the blister and input port hole; and

6. Example of an integrated PCR diagnostic cartridge with one or more packaged blisters to realize a self-sufficient diagnostic cartridge

The below discussion describes exemplary methods of manufacture and use of blister packaging. Additional fabrication techniques and applications are within the scope of one of skill in the art.

### A. Cold-Forming a Blister

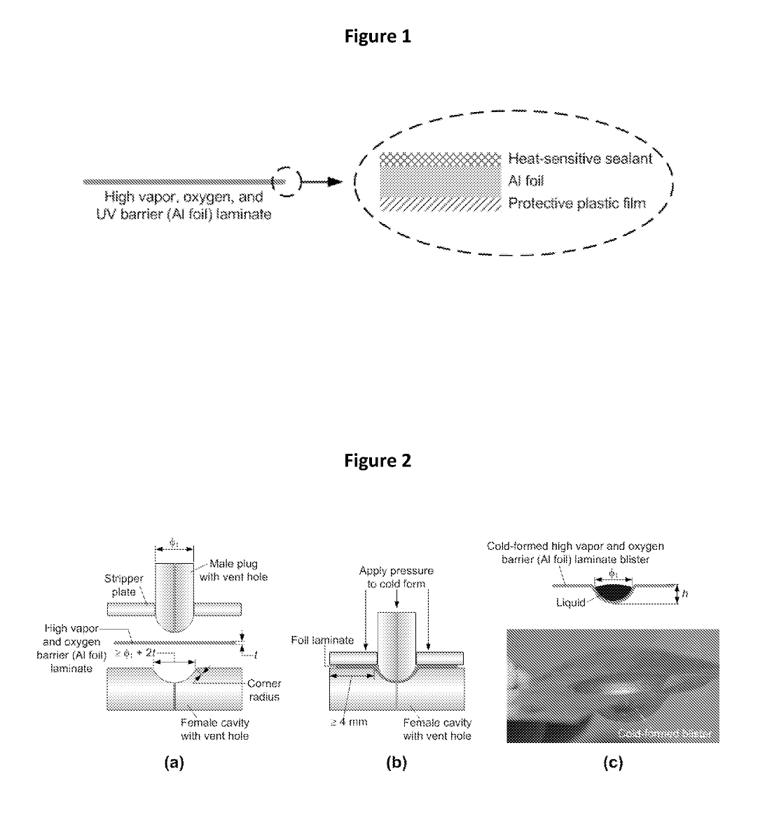

In some embodiments, pressure (cold) formable high vapor, oxygen, other gases, and UV barrier laminates are chosen and used to create blisters into which liquids are stored. The option of choosing materials that can be cold-formed presents the advantage of lowering the production cost since heat (for thermoforming applications) is not required. These high vapor and oxygen barrier laminates can be manufactured to be transparent or opaque. Transparent laminates offer almost equal barrier protection through numerous methods, such as, for example SiOₓ and Al₂O₃, and many can be either cold- or thermo-formed; however, the transparent laminates cost 4-10× as much as opaque laminates that alternatively use a thin sheet of aluminum (Al) foil to serve as a barrier. To reduce the overall cost of the disposable plastic diagnostic cartridge, some embodiments of the present invention use opaque Al or other metal foil laminates. The total thickness of such laminate films typically ranges from 0.002″ to 0.012″. In general, they are comprised of at least three laminates—heat-sensitive sealant, Al foil film, and plastic film to protect the Al foil from physical damage (tears, scratches) (See e.g., FIG. 1).

In some embodiments, the blisters are formed in the laminate using a cold-forming station that consists of a male plug, stripper plate, and female cavity. The heat-sensitive sealant side of the laminate film, which is chosen to be compatible with liquid to be stored within it, faces the male plug for the subsequent heat sealing process.

FIG. 2 shows a process diagram of one method of how a blister can be cold-formed in a high vapor and oxygen barrier laminate. The laminate film is held firmly using applied pressure between the female cavity and stripper plate, both of which are machined to a very flat and uniform surface. A minimum of 0.157″ (4.0 mm) from the edge of the female cavity is preferably held firmly flat to allow for the heat sealing process in order to prevent the laminate from wrinkling. Next, with applied pressure, the male plug subsequently comes down on the laminate film, creating a hemispherical blister. The dimensions of the male plug and female cavity are designed according to the amount of liquid that needs to be stored in the blister. The diameter of the female cavity is ≧φ₁+2t, where φ₁ is the diameter of the male plug, and t is the thickness of the foil laminate sheet. The depth of the cold-formed blister (h) is dependent on how far the male plug is pushed into the laminate sheet and affects the total liquid capacity of the blister. The applied pressure for both the stripper plate and male plug can be realized by many different schemes, including, but not limited to, by manual compression using screws, compressed air, and stepper motors.

The shape of the blister is dependent on the shapes of the male plug and female cavity, and is not limited to the hemispherical shape (e.g., oval, square, rectangular, etc.). In some embodiments, a chamfer (corner radius) is machined in the female cavity to prevent tearing/pinching of the laminate film at the edge. In some embodiments, to prevent stiction of the laminate film to the male plug or female cavity during forming, very small vent holes are drilled into both the male plug and female cavity. The vent holes allow air to escape during the cold forming and prevent any vacuum build-up.

In one exemplary embodiments, the final aspect ratio h/Φ₁ of the blister, as shown in FIG. 2(c), is ≧0.30. No tears or pinholes are visually observed in the laminate sheet after cold-forming.

In some embodiments, blisters are designed with a head space to allow blisters to move along a conveyor, as they would be in a full line production facility using a form/fill/seal (F/F/S) machine. This significantly reduces any chances of spilling the liquid over the brim of the blister edge. In a F/F/S system, a web of blisters will be moving down the line, accelerating and decelerating, which may cause the liquid to potentially spill over. Embodiments of the present invention overcome such issues by providing dead space in blisters.

In some embodiments, the assay cartridge comprises one or more (e.g., two) alignment pins that help to secure the blister to the cartridge.

B. Liquid Aliquoting into a Cold-Formed Blister

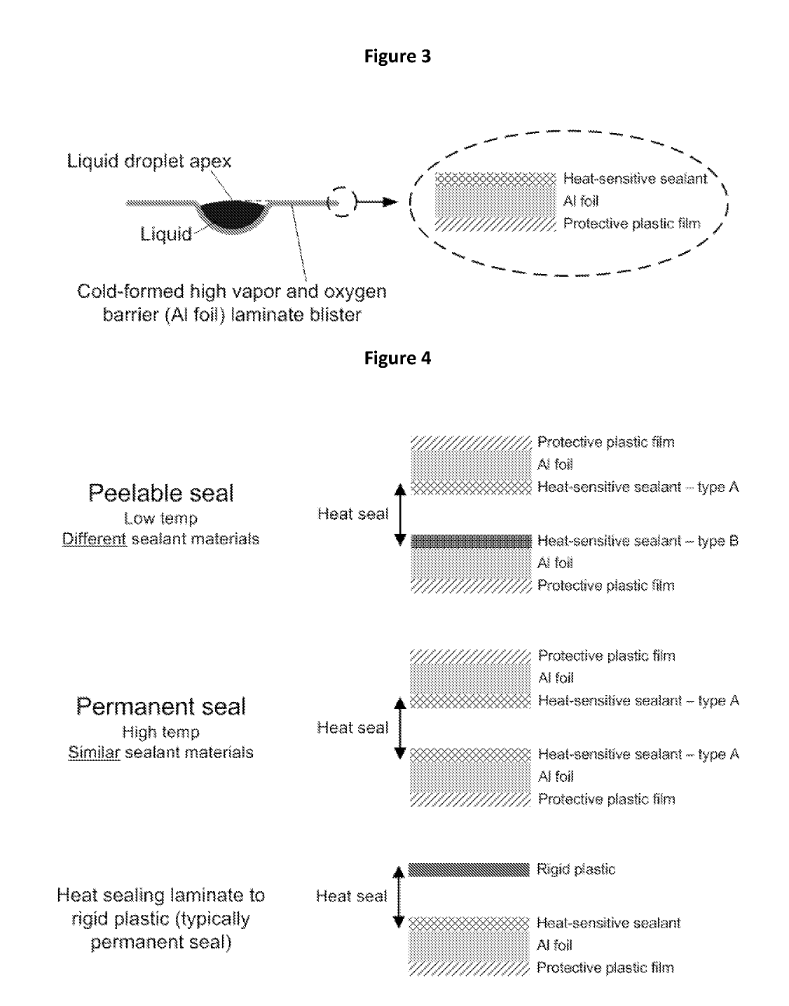

In some embodiments, once the blister has been cold-formed, liquid can be aliquoted into it manually (e.g., using a pipetting device) or automatically using a liquid dispensing tool (e.g., during manufacturing). In one exemplary embodiment, a manual pipetting device (e.g., PIPETMAN pipetting device (0.1 μL resolution)) is used to deposit the desired liquid volume, which ranges from 10 μL to 1.0 mL. The heat-sensitive sealant film on the laminate sheet is often hydrophobic, causing aqueous liquids to have a relatively high contact angle. Here, liquid is filled in the blister until the apex of the liquid droplet aligns with the top of the laminate sheet as shown in FIG. 3.

In is preferred that the apex of the liquid droplet should not be higher than the top of the foil laminate since in the subsequent heat sealing procedure, the liquid may spill/leak out of the blister and into heat sealing areas (i.e., perimeter of the blister). This is especially usefull for non-aqueous liquids since they will tend to wet the surface of the blister, creating a small contact angle compared to aqueous liquids. The heat-sensitive surface of the blister may also be surface treated by various chemical or physical methods, such as, for example, surfactants or plasma, to increase its hydrophilicity (suitable for aqueous liquids). Treatment methods are designed to not affect the heat sealing qualities of the sealant film. In some embodiments, the aqueous liquid contains a chemical component (e.g., surfactant or detergent) that makes it preferentially wet the blister surface by reducing its surface tension.

### C. (Heat) Sealing the Blister to Store the Liquid

One of any number of technologies to bond/seal materials (e.g., laminate to laminate, laminate to rigid plastic, plastic to plastic) with heat-sensitive sealants may be utilized, including but not limited to constant heat sealers, impulse heat sealers, laser welding, radio frequency, and ultrasonic sealing. The exemplary method described here is based on using an impulse heat sealer, in which a mechanical pressure is first applied to the perimeter of the blister to sandwich both laminate films together, creating a liquid and vapor tight closure. Next, the power to the heat seal band is turned on, rapidly increasing the heat and melting the heat-sensitive sealants and bonding them together. The heat is turned off, but the mechanical clamping pressure is only released once the heat seal band has cooled, and the seal has set and has good strength and appearance. The advantages of this method, compared to a constant heat sealer where the heat is always on, are:

(1) a stronger seal is created with superior appearance, and (2) the liquid is not exposed to high temperatures during the heat sealing process, which can cause liquid evaporation, and vapor entrapment in the heat seals (poor seal strengths).

A heat sealer is a function of four parameters:
Time—length of time the heat seal bands maintained at the preset temperature which depends on several parameters, including thickness of the laminate sheets and/or rigid plastics and type of seal strength desired.
Pressure—the amount of pressure (psi) exerted down on the two materials to be sealed together (e.g., laminate to laminate, laminate to rigid plastics).
Temperature—the temperature of the heat seal band which generally ranges from 200-500° F.
Heat-sensitive sealant material—are the sealant materials for both laminate sheets similar or different.

Two types of seals can be created by manipulating the combination of the aforementioned parameters: (1) peelable seals which have a lower peel strength and are designed to be opened either manually by hand, or with the assistance of an automated machine. They are created at low temperatures with dissimilar heat-sensitive sealant materials. A variety of heat-sensitive sealants are available from manufacturers specifically designed to fabricate peelable seals. (2) permanent seals which have significantly stronger peel strength and are not designed to be opened. These are created at high temperatures with similar heat-sensitive sealant materials. FIG. 4 shows a conceptual diagram of how peelable and permanent heat seals are made by choosing the appropriate heat sealing temperature and sealant material. In some embodiments, a laminate sheet can be heat sealed to a rigid plastic material with similar material properties.

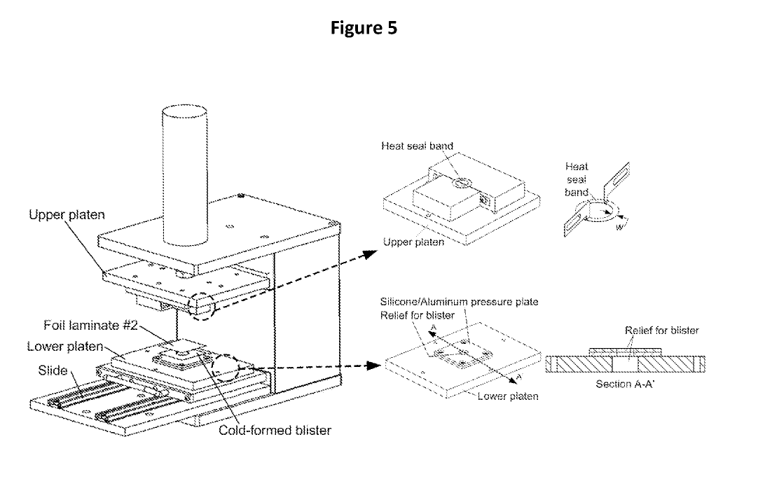

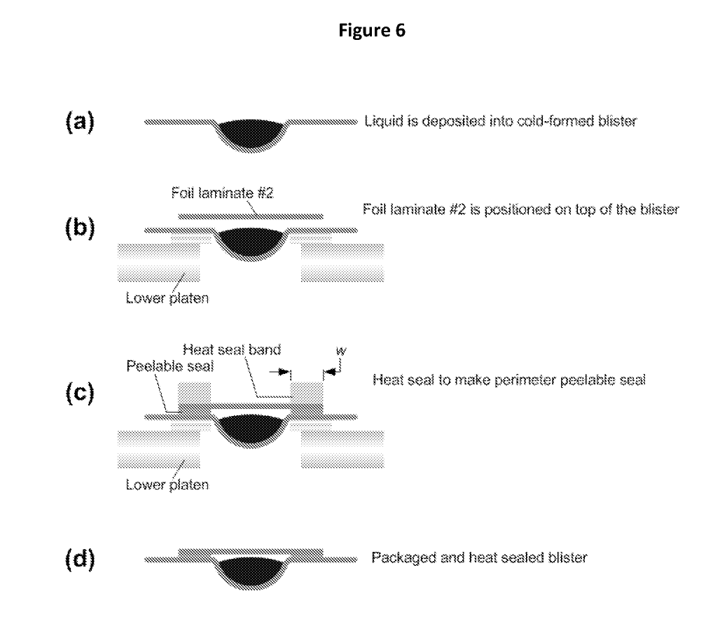

In some embodiments, these processes are used to heat seal and store the liquid inside the cold-formed blister. In some embodiments, peelable seals are created to allow subsequent bursting of the blisters when the liquids are required for use. In some embodiments, a contour impulse heat seal press with an upper and lower platen and remote user-operated module (to configure the time, temperature, and pressure synchronization) is designed and developed to make the heat seals (see FIG. 5). The upper platen holds an interchangeable circular heat seal band (band width, w) that creates a circular perimeter seal around the blister. The heat seal band is designed to match the blister geometry. The larger w, the greater the seal strength, but at the cost of requiring more power and force by the press to create the seal. Typical values for w for heat sealing applications range from ⅛″ to ¼″. The lower platen holds interchangeable silicone/aluminum pressure plates that are machined to a specific blister geometry diameter. A relief is provided in both the pressure plate and platen to accommodate for the blister and prevent it from crushing during the heat sealing process. The general heat sealing process used in exemplary embodiments to create peelable seals is schematically shown in FIG. 6.

In some embodiments, the cold-formed blister previously filled with the liquid is positioned on the lower platen, cradled by the silicone/aluminum pressure plate and relief. Foil laminate #2, which has a different sealant specifically designed for peelable seals, is positioned on top so that the heat sensitive sealant faces down (similar to the top image in FIG. 4) to facilitate heat sealing. The upper platen comes down with a force, mechanically clamping the blister. Power to the heat seal band is turned on to its preset temperature for a brief time period until a heat seal is realized. The power is turned off to the heat seal band, and when it has cooled, the upper platen is raised to release the mechanical clamping. This results in a packaged and heat sealed blister with peelable seals that is ready to be integrated with a rigid (plastic) cartridge.

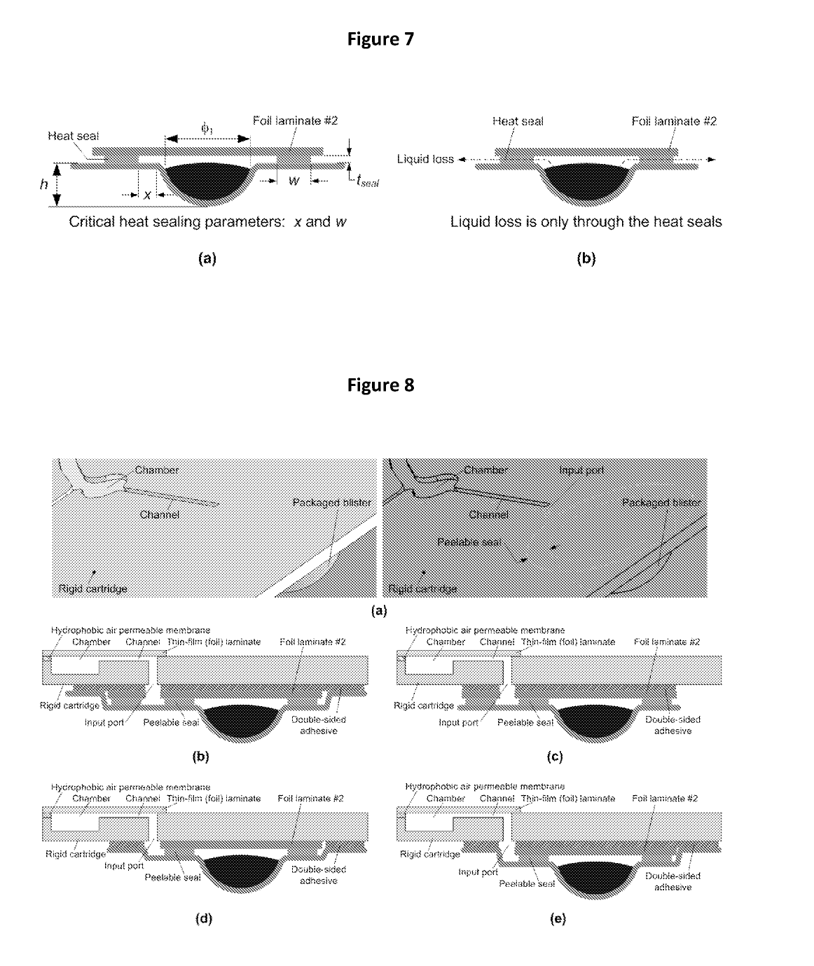

FIG. 7 shows cross-sectional diagrams of a packaged blister. Heat seal parameters, in addition to the aforementioned blister geometries, are indicated in FIG. 7(a). x is the distance between the edge of the cold-formed blister and where the heat seal begins. Here, it is minimized as much as possible (≦0.01″). As mentioned previously, w is the heat seal width created by the heat seal band. Some trapped air (which is compressible) in the packaged and sealed blister is necessary so that external air pressure changes (e.g., during air shipment) will not cause the blister to collapse or burst. Similarly, it is beneficial to minimize the trapped air to both reduce the overall size of the blister and dead volume spaces where the liquid can become trapped during bursting.

FIG. 7(b) shows a similar cross-sectional diagram that describes how and where liquid loss can occur over time. Due to the Al foil in both foil laminates, liquid loss does not occur through the laminate sheets, but only through the heat sealed sealants. This liquid loss is a function of blister volume, ambient environmental temperature, heat-sensitive sealant material properties (permeability), heat seal surface area (a function of w), and tₛₑₐₗ—the thickness of the final heat seal.

D. Integration of a Packaged Blister with a Rigid Cartridge

In some embodiments, heat sealed blisters are integrated with an assay device (e.g., rigid cartridge). In some embodiments, polypropylene plastic is chosen as the material for the cartridge. Polypropylene is cost effective and safe and (bio)compatible for the diagnostic chemistry and readily disposable. The plastic cartridge is manufactured using one of many existing high-volume techniques, including, but not limited to, injection molding and vacuum forming. The blister is then integrated with the rigid cartridge. In some embodiments, (1) double-sided adhesives are used, while in other embodiments, (2) sealing via impulse/constant heat sealers, laser welding, radio frequency, or ultrasonic methods are used. Both techniques are described here.

In some embodiments, when using double-sided adhesives, one of three methods is selected for bonding the packaged blister to the rigid cartridge. Conceptual perspective and cross-sectional diagrams of the integrated blister and cartridge are shown in FIG. 8.

FIG. 8(a) shows conceptual solid (left) and semi-transparent (right) perspective views of the blister integrated with the rigid cartridge. The cartridge has an input port, channel, and chamber, which are made accessible to the packaged blister. A hydrophobic air permeable membrane is also integrated into the cartridge to allow air to escape when the blister is burst and liquid fills the cartridge channel and chamber. The blister is positioned just outside the input port to the cartridge channel and chamber. FIG. 8(b-e) show corresponding cross-sectional diagrams of four exemplary methods by which the blister can be bonded to the cartridge using double-sided adhesives (not drawn to scale). FIG. 8(b) shows a blister with an extended foil laminate #2 with a slit that is subsequently aligned with the cartridge's input port. Double-sided adhesive, which also has a slit for the input port, is used to bond the entire surface area of the blister to the cartridge. This is advantageous since the liquid will only flow on the foil laminate (minimal contact if any with the adhesive) and also ensure there are no air gaps between the blister and cartridge where the liquid may potentially wick into after bursting. FIG. 8(c) shows a slight modification to FIG. 8(b) where the cold-formed blister laminate is not adhesively bonded to the rigid cartridge. FIG. 8(d-e) shows a blister with foil laminate #2 dimensionally trimmed to the blister geometry. Once the blister is burst, the liquid will again have minimal, if any, contact with the adhesive; however, there may be a potential for the liquid to wick between foil laminate #2 and rigid cartridge. This may either be avoided due to the (flat) pressure that is applied to burst the blister (See below section), or by modifying the design to FIG. 8(e).

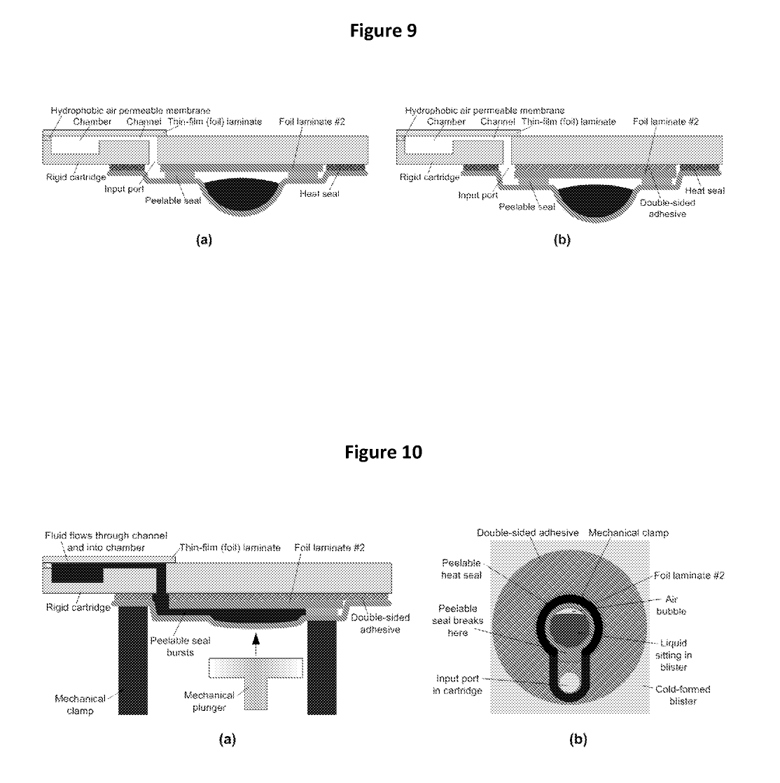

An alternative method of bonding the blister to the rigid cartridge is primarily using heat seals, with the option of adding some double-sided adhesive. Cross-sectional diagrams of this method are shown in FIG. 9.

FIG. 9(a) shows how a packaged blister can be bonded to a rigid cartridge using heat seals. Here, the heat-sensitive sealant material has closely matching properties with the cartridge material, and therefore can be designed to be a permanent seal (higher peel strength compared to a peelable seal). FIG. 9(b) shows an alternative method by which a combination of heat seals and double-sided adhesive tape is used to bond the packaged blister to the cartridge. This reduces the chance of any air pockets that may trap the liquid once the blister has been burst. However, it also introduces the potential for the liquid to come in contact with the adhesive as it flows into the cartridge chamber via the channel and input port.

### II. Embodiments of the Invention in Use

In some embodiments, the present invention provides methods of performing assays using the liquid storage, assay and seal bursting devices described herein. The present invention is not limited to the exemplary systems and methods described below.

### A. Bursting the Blister and Delivering the Liquids

When the diagnostic cartridge is ready to be used, the liquid-containing blisters are burst open, and the liquid is directed into the cartridge chamber via the channel and input port. In some embodiments, a seal bursting component is utilized to burst the seals and deliver the liquid to an assay device. Two model integrated cartridge and blister systems are shown in FIG. 8(e) and FIG. 9(a). There are two possible bursting mechanisms by which the blister can be burst—(1) in conjunction with FIG. 8(e), apply a mechanical clamp around the periphery of the blister and input port to specify the burst site on the peelable seal and ensure the liquid only flows towards the input port, and (2) in conjunction with FIG. 9(b), there is no mechanical clamp, but only the plunger which bursts the blister; the difference in peelable and permanent peel strengths is leveraged. Both of these bursting mechanisms are described in detail below.

FIG. 8(e) shows a model of how the peelable seal is burst with the aid of a mechanical clamp to deliver the liquid inside the cartridge chamber. FIG. 10 shows cross-sectional and top-view diagrams of a ruptured peelable seal.

A mechanical clamp is aligned and pressed against the rigid cartridge on the peelable heat seals and around the input port in the cartridge as shown in FIG. 10(b). The purposes of the mechanical clamp are: (1) to provide a leak-proof seal so the liquid will only flow in the specified areas, (2) specify a consistent location where the peelable seal will burst. A mechanical plunger subsequently applies uniform and controlled pressure against the packaged blister until the peelable seal bursts. The plunger regulates the amount of pressure against the packaged blister to control the liquid volume that flows out of the burst blister, through the input port, and into the cartridge chamber via the channel—see FIG. 10(a). The cartridge is positioned vertically so that the trapped air bubble in the blister will rise to the top, opposite to the input port. This bursting mechanism allows a user to consistently know how and where the blister will burst and liquid will flow. It also allows a user to compensate for the presence of the trapped air bubble in the cold-formed blister, and ensure the location of the burst occurs where there is no air bubble, (bubbles rise to the top) but only liquid. The cartridge is filled only with liquid; any air bubbles that may be introduced into the cartridge will rise to the top and exit via the hydrophobic air permeable membrane. Furthermore, the clamp minimizes the potential dead air spaces where the liquid could potentially migrate to, minimizing the dead (liquid) volume. This saving on liquid loss can reduce the initial liquid fill volume and blister size and geometry, saving on material cost.

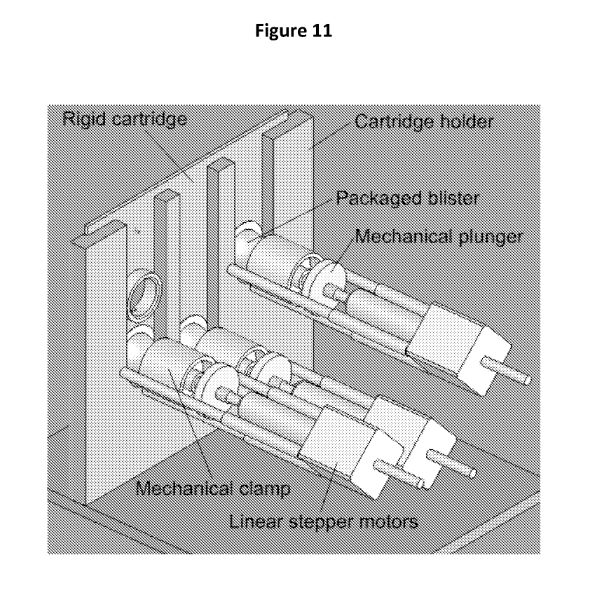

A diagram of one exemplary method of how a cartridge may interface with the mechanical clamp and plunger is shown in FIG. 11. In some embodiments, the rigid cartridge has multiple (e.g., two or more, three or more, four or more, etc.) packaged blisters that is held in place by a cartridge holder. Separate mechanical clamps and plungers are appropriately dimensioned to match the blister geometry and are individually driven by a linear stepper motor. In some embodiments, a single linear stepper motor is used to drive multiple clamps and plungers. The mechanism to drive these mechanical modules is not limited to a stepper motor, but also extends to any other mechanism which creates controllable and consistent force outputs. The mechanical clamps and plungers align with the blisters that have been previously bonded to the rigid cartridge and aligned with their respective input ports.

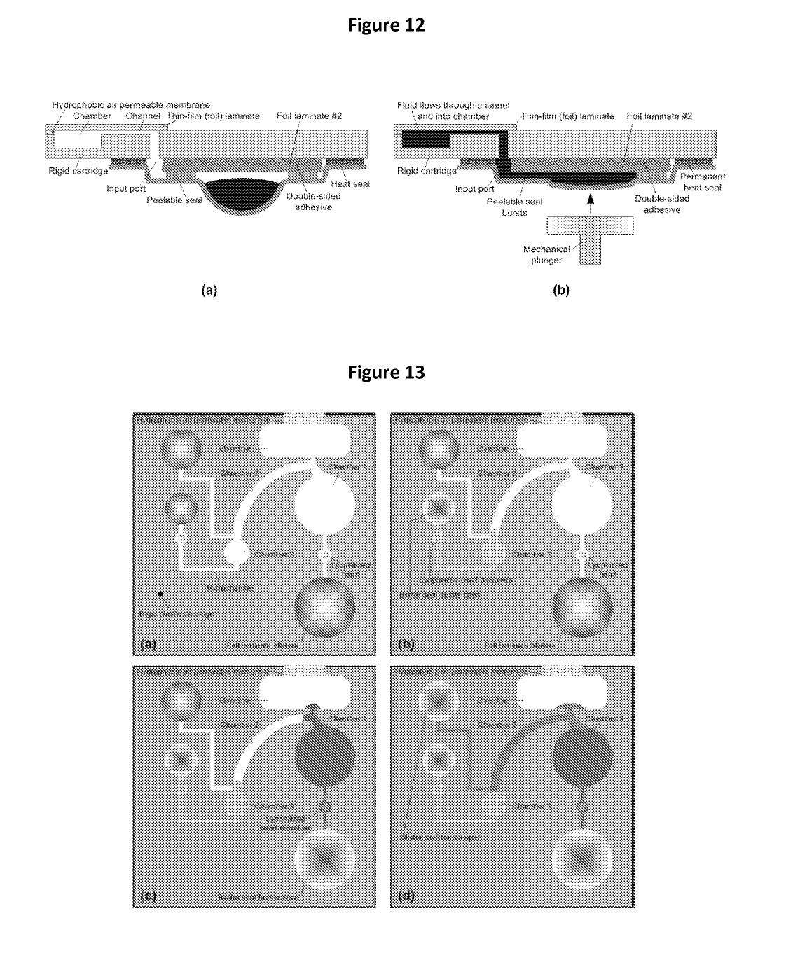

The second method of bursting leverages the peel strength differences between peelable and permanent heat seals. This mode works with the integrated blister and cartridge designs, for example, as shown in FIG. 9. Due to the differences in the peel strengths between the peelable seal, which stores the liquid in the blister, and permanent seal that bonds the blister to the rigid cartridge, it is possible to remove the mechanical clamp altogether. FIG. 12 shows an exemplary cross-sectional diagram of how the integrated cartridge and blister from FIG. 9(b) is aligned and coupled with the mechanical plunger.

When the mechanical plunger is pressed against the cold-formed blister, the peelable seal bursts, allowing the liquid to flow into the cartridge via the input port. The permanent heat seal remains intact, preventing the liquid from leaking outside the cartridge module.

In some embodiments, a blister crusher module is designed to crush multiple blisters that are adhesively bonded to the plastic microfluidic assay cartridge (see FIG. 13(c)). The number of blisters can be adjusted according to the assay requirements. The blister crusher crushes the blisters by peeling the peelable seal in a specific location, and dispensing the liquid into the cartridge channel and chamber via an input port. See FIG. 13.

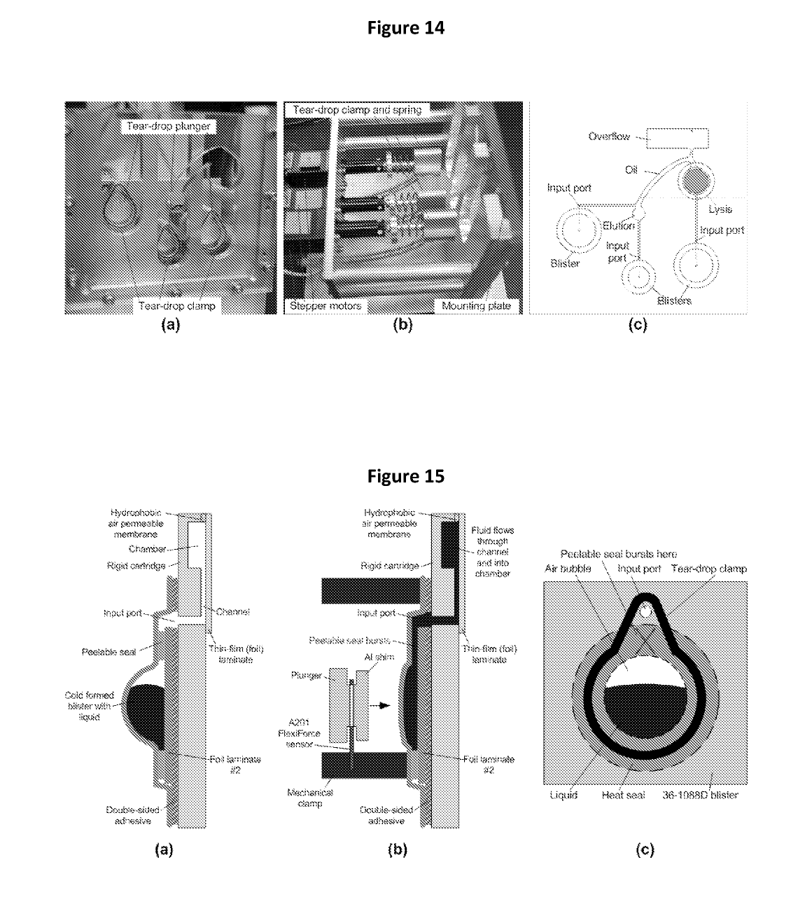

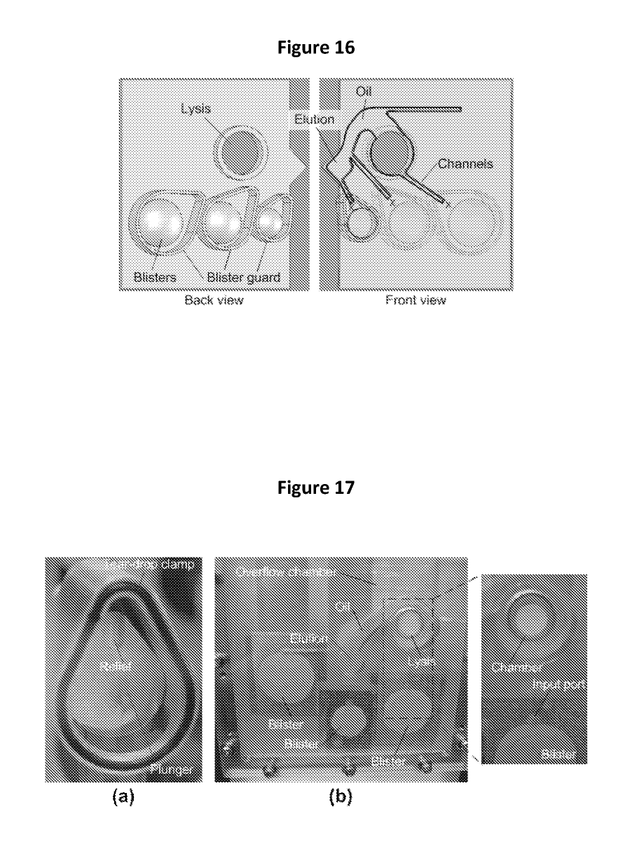

In some embodiments, tear-drop clamps are used to direct the peeling of the peelable heat seal on the blister so that the bursting consistently occurs in a pre-designated location. See FIG. 14. In some embodiments, a blister is adhesively bonded to the surface of the cartridge or bonded using other sealing techniques and positioned such that the top of the heat seal perimeter is directly below the input port (FIG. 14(c)). Therefore the orientation of the cartridge is important when crushing the blisters (see FIG. 16(b)). The tear-drop clamps apply uniform pressure across most of the heat seal perimeter, except for the top. The applied pressure should be large enough to compensate for any differences in the heat seal quality so that the bursting will have a natural tendency to always peel at the top. This ensures that once the heat seal is compromised, as shown by the x in FIG. 14(c), the air will escape first, followed by the liquid. In some embodiments, the cartridge is provided with a small exit port hole inside the ‘overflow’ chamber, which allows liquids from the blisters to enter the cartridge channels. The air-liquid sequence is particularly important since crushing the blister where the liquid comes out first (i.e., the blister is positioned so that the bottom of the heat seal perimeter is above the input port) causes a heavy mixture of air bubbles and liquid to be dispensed into the cartridge.

In some embodiments, the clamping mechanism is integrated directly into the disposable microfluidic cartridge. For example, once the blisters are bonded to the cartridge, smaller pieces of plastic materials (blister guards) mate with the microfluidic cartridge and provide the necessary mechanical clamping pressure to ensure the blister heat seal only bursts in one location (as shown by the x in FIG. 15).

In some embodiments, plungers for bursting blisters are shaped similar to a tear-drop shape and are sized slightly larger than the blister to ensure it covers the entire surface area for bursting. This ensures complete compression of the blister and minimizes dead (trapped) liquid volume inside the blister that does not get dispensed into the cartridge. In some embodiments, plungers have a small channel relief cut into the top which prevents complete closure of the channel that is formed during crushing (i.e., when the peelable heat seal peels apart) which allows the liquid to move from the blister into the input port and cartridge.

In embodiments that utilize multiple blisters the liquids are burst in a specific sequence to prevent any cross-contamination, especially between the lysis and elution chambers. Furthermore, the liquid dispensing ensures that no air bubbles are in the channel and chamber network since they could interfere with the subsequent assay processing. There are two exemplary sequence methods that may be used.

Method 1
1. Burst the elution blister and fill the channel and chamber.
2. Burst the lysis blister and fill the chamber, ensuring it does not overflow.
3. Burst the oil blister to fill the channel and chamber gaps between the elution and lysis. Since it is an immiscible liquid, it will prevent cross-contamination between the lysis and elution reagents.

Method 2
1. Burst the elution blister and fill the channel and chamber.
2. Burst the oil blister to fill the channel and part of the chamber. This will create a liquid buffer between the elution and lysis, perchance the lysis blister overfills and flows into the oil chamber and subsequently into the elution chamber.
3. Burst the lysis blister and fill the channel and chamber.
4. Dispense additional oil from the already-crushed oil blister to fill the remaining chamber gaps between the lysis and elution reagents.

In some embodiments, the method by which the cartridge is designed, and its vertical orientation (which facilitates use of gravity), allow for any air bubbles that have entered the cartridge to float up to the top and near or into the overflow chamber.

### B. Exemplary System

FIG. 13(a) shows an exemplary integrated cartridge that is designed for a diagnostic assay (e.g., PCR assay), with multiple packaged blisters that contain distinct aqueous and/or non-aqueous liquids, in the volume range of 0.10 mL to 1.0 mL. The embodiment also shows integration of lyophilized assay pellets that are dissolved once a blister is burst and the liquid passes through the input port, fluid channel and into the respective chamber. In some embodiments, An overflow chamber which contains the hydrophobic air permeable membrane is also integrated into the cartridge to allow passage of air when filling the chambers with liquid. One by one, or simultaneously, depending on the application, a blister is burst, dispensing its stored liquid into the rigid cartridge—see FIG. 13(b-d). Since the cartridge is kept in the upright position, as shown in the diagrams, any air bubbles that may be transferred from the blister to the rigid cartridge easily float up to the top of the channels and chambers, and into the overflow chamber. Here, the liquid filling chamber 2 is a nonaqueous liquid that helps prevent any contamination by separating the liquid in chamber 3 from both chamber 1 and overflow (U.S. Pat. No. 6,103,265; herein incorporated by reference). In some embodiments, the scheme of bursting and filling the cartridge chambers with the appropriate liquids is fully automated, as previously described by FIG. 11.

In some embodiments, the cartridge is positioned vertically so that the liquid inside the blister is pulled down by gravity, as shown in FIG. 14(a). This also ensures that once the heat seal is burst open, the air will first escape, followed by the liquid. This minimizes the number of air bubbles injected into the cartridge channel and chambers. The inset image in FIG. 16(b) shows how the blisters are positioned directly beneath the input port.

### C. Applications

The systems and methods of embodiments of the present invention find use in a any number of diagnostic assays. Examples include, but are not limited to, PCR medical diagnostics tests (e.g., for infectious diseases such as HIV). In some embodiments, the systems and methods of the present invention find use in performing assays in resource limited areas where temperature controlled environments may not be available. In some embodiments, assays are packaged as self-sufficient, individual tests that will have all the necessary (liquid) reagents on-cartridge to complete the patient's analysis. By further integration with lyophilized assay beads, cold chain technology is avoided, saving on cost and making the test more robust and readily available to a larger public.

The systems and methods of embodiments of the present invention have numerous benefits and applications in any lab-on-a-chip technology where relatively small amounts of liquids must be stored with the test cartridge. Examples of research and diagnostic assays suitable for use with the systems and methods described herein are described below.

i. Sample

Any sample suspected of containing the desired material for purification and/or analysis may be tested according to the disclosed methods. In some embodiments, the sample is biological sample. Such a sample may be cells (e.g. cells suspected of being infected with a virus), tissue (e.g., biopsy samples), blood, urine, semen, or a fraction thereof (e.g., plasma, serum, urine supernatant, urine cell pellet or prostate cells), which may be obtained from a patient or other source of biological material, e.g., autopsy sample or forensic material.

Prior to contacting the sample with the device or as a component of the device or automated system, the sample may be processed to isolate or enrich the sample for the desired molecules. A variety of techniques that use standard laboratory practices may be used for this purpose, such as, e.g., centrifugation, immunocapture, cell lysis, and nucleic acid target capture.

In other embodiments, the methods of embodiments of the present invention are utilized to purify and/or analyze intact cells (e.g., prokaryotic or eukaryotic cells).

ii. Nucleic Acid Detection

Examples of nucleic modification/analysis/detection methods include, but are not limited to, nucleic acid sequencing, nucleic acid hybridization, and nucleic acid amplification. Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing. Illustrative non-limiting examples of nucleic acid hybridization techniques include, but are not limited to, in situ hybridization (ISH), microarray, and Southern or Northern blot. Nucleic acids may be amplified prior to or simultaneous with detection.

Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

Non-amplified or amplified target nucleic acids can be detected by any conventional means. For example, target mRNA can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and many types of interacting label pairs are known (e.g., U.S. Pat. No. 6,534,274, herein incorporated by reference in its entirety).

Another example of a detection probe having self-complementarity is a “molecular beacon” (see U.S. Pat. Nos. 5,925,517 and 6,150,097, herein incorporated by reference in entirety). Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS).

Other self-hybridizing probes are well known to those of ordinary skill in the art. By way of non-limiting example, probe binding pairs having interacting labels (e.g., see U.S. Pat. No. 5,928,862, herein incorporated by reference in its entirety) may be adapted for use in the compositions and methods disclosed herein. Probe systems used to detect single nucleotide polymorphisms (SNPs) might also be used. Additional detection systems include “molecular switches,” (e.g., see U.S. Publ. No. 20050042638, herein incorporated by reference in its entirety). Other probes, such as those comprising intercalating dyes and/or fluorochromes, are also useful for detection of amplification products in the methods disclosed herein (e.g., see U.S. Pat. No. 5,814,447, herein incorporated by reference in its entirety).

In some embodiments, detection methods are qualitative (e.g., presence or absence of a particular nucleic acid). In other embodiments, they are quantitative (e.g., viral load).

iii. Protein Detection

Examples of protein detection methods include, but are not limited to, enzyme assays, direct visualization, and immunoassays. In some embodiments, immunoassays utilize antibodies to a purified protein. Such antibodies may be polyclonal or monoclonal, chimeric, humanized, single chain or Fab fragments, which may be labeled or unlabeled, all of which may be produced by using well known procedures and standard laboratory practices. See, e.g., Burns, ed., Immunochemical Protocols, 3^{rd} ed., Humana Press (2005); Harlow and Lane, Antibodies: A Laboratory Manual, Cold Spring Harbor Laboratory (1988); Kozbor et al., Immunology Today 4: 72 (1983); Köhler and Milstein, Nature 256: 495 (1975). In some embodiments, commercially available antibodies are utilized.

### D. Data Analysis

In some embodiments, following purification and detection, a computer-based analysis program is used to translate the raw data generated by the detection assay (e.g., the presence, absence, or amount of a given target molecule) into data of predictive value for a clinician or researcher. In some embodiments, the software program is integrated into an automated device. In other embodiments, it is remotely located. The clinician can access the data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

Any method may be used that is capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw data, the prepared format may represent a diagnosis or risk assessment (e.g., viral load levels) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

### E. Compositions & Kits

In some embodiments, systems and/or devices of the present invention are shipped containing all components necessary to perform purification and analysis (e.g., blister seals reagents and cartridges for performing assays). In other embodiments, additional reaction components are supplied in separate vessels packaged together into a kit.

Any of these compositions, alone or in combination with other compositions disclosed herein or well known in the art, may be provided in the form of a kit. Kits may further comprise appropriate controls and/or detection reagents. Any one or more reagents that find use in any of the methods described herein may be provided in the kit.

### EXPERIMENTAL

The following examples are provided to demonstrate and illustrate certain preferred embodiments and aspects of the compositions and methods disclosed herein, but are not to be construed as limiting the scope of the claimed invention.

### Example 1

### Bursting Force

Experiments were conducted to ascertain how much force is required to burst open blisters. This information is used to aid in determining the correct type of stepper motor (which outputs sufficient force to depress the plunger against the blister). An Instron compression instrument was used to blindly burst open the blisters (i.e., no tear-drop clamps). The blister parameters are described below.

Lysis blisters
0.72″ diameter
0.20″ stroke (depth)
Liquid volume=500 μL

Oil blisters
0.72″ diameter
0.18″ stroke (depth)
Liquid volume=400 μL

Elution blisters
0.55″ diameter
0.150″ stroke (depth)
Liquid volume=150 μL

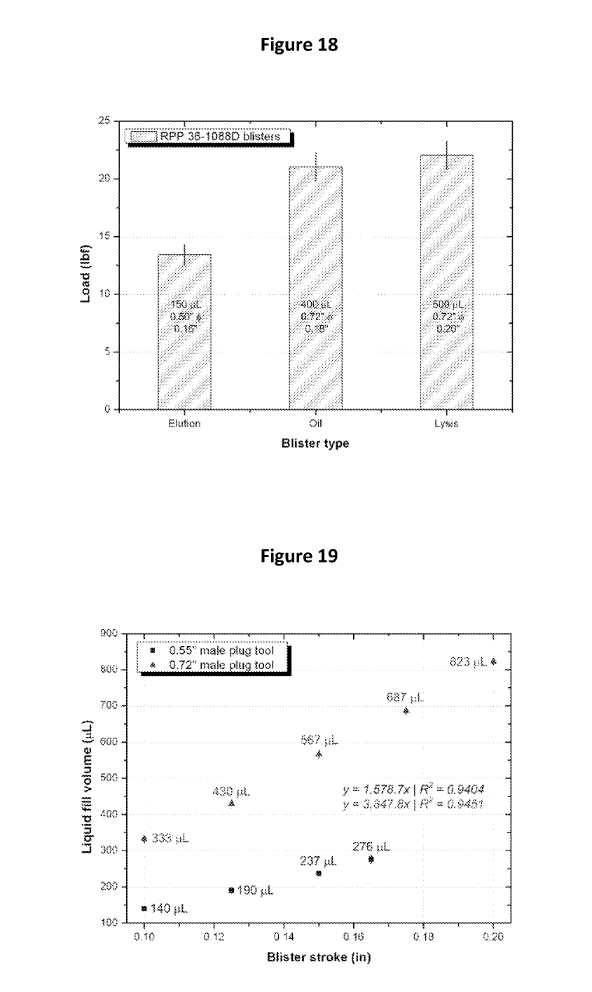

The graphical data is shown in FIG. 18. The data plot shows the average load (force) required to burst open the elution, oil, and lysis blisters. The relatively consistent forces, as seen by the relatively narrow standard deviations, indicate that the heat sealing quality is consistent. The forces will change if either the blister size, seal width or liquid volume inside the blister is changed.

### Example 2

### Accelerating Aging Experiments

Experiments were conducted to quantify the heat sealing process which bonds the two foil laminates together. The quality of the heat seal has a direct impact on both the force required to burst open a blister and any liquid loss via evaporation. The blisters were stored in a forced convective oven at 42-45° C. (1-3% RH) for several weeks. Furthermore, to simulate cold shipment transportation, the blisters were exposed from room temperature (RT) to 0° C. for 16 hours, 0° C. to −20° C. for 8 hours, −20° C. to 0° C. for 16 hours, and back to RT. The liquid loss was measured through periodic weight measurements. Design of Experiments (DOE) were performed with each liquid reagent (elution, lysis, and oil) to determine the optimal time and temperature regime. It was previously determined that pressure, in the range of 50-90 psi has little or no impact on the quality of the heat seal. Blisters with no liquids were also heat sealed to determine any physical change in the blister material itself that would lead to a change in weight. This serves as the baseline for weight loss observed in blisters with liquids inside.

Lysis DOE

Time=2, 5, 8 s

Temperature=191, 211, 232° C.

Elution DOE

Time=2, 5 s

Temperature=191, 211° C.

Oil DOE

Time=2, 5 s

Temperature=191, 211° C.

While no evaporation is expected for oil blisters, the oil may have wicked onto the heat seal surface, compromising the quality of the heat seal. Also, it is desirable to determine a universal time and temperature that would work for all three blisters and liquids since it would be extremely helpful in large-scale manufacturing. The weight loss data showed the following observations:

Lysis blisters

49 days

Empty blister average weight loss and standard deviation=0.0006 g±0.0001

Liquid blister weight loss varied between 0.0005-0.0013 g

Observation—taking the empty blister weight loss into consideration, the actual liquid loss was approximately 0.0006 g at most, which corresponds to 0.6 μL and indicates very good heat seals

Elution Blisters

36 days

Empty blister average weight loss and standard deviation=0.0002 g±0.0001

Liquid blister weight loss varied between 0.0001-0.0003 g

Observation—taking the empty blister weight loss into consideration, the actual liquid loss is inconsequential, which indicates very good heat seals

Oil Blisters

20 days

Empty blister average weight loss and standard deviation=0.0001 g±0.0001

Liquid blister weight loss varied between 0.0000-0.0005 g

Observation—taking the empty blister weight loss into consideration, the actual liquid loss was approximately 0.0003 g at most, which corresponds to 0.38 μL and indicates very good heat seals

### Example 3

### Liquid Volume Fill Capacity

Experiments were conducted to determine the total liquid volume fill capacity for a given blister. This is dependent on the blister diameter and stroke (depth). This information is used to determine the maximum amount of liquid than can be safely heat sealed inside a blister without overflowing (i.e., onto the heat seal perimeter) and compromising the heat seal process. This is especially useful for liquids that preferentially wet the surface of the foil laminate blister. Two blister diameters were characterized: 0.55″ and 0.72″. Multiple blisters with varying strokes (starting at the maximum stroke where the foil laminate did not tear and decreasing the depth progressively) were cold formed and heat sealed empty. A fine gauge needle was used to pierce the lidstock foil laminate and dispense liquid inside the empty blister until liquid started to spill out. This was determined to be the liquid volume fill capacity for a blister. See FIG. 19.

The data plot shows the liquid volume fill capacity for both blister diameters at several stroke values. The maximum volume is listed above each data point. A linear trend line was also determined for each blister diameter that can offer additional numerical interpretation at other stroke values.

### Example 4

### Dead Volume

When a blister is crushed completely, a percentage of the liquid will always remain inside the blister due to how the blister is crushed—the creases can trap small amounts of liquids. It is useful to characterize the dead volume since it relates directly to the volume that should be inside the blister (blister liquid volume=channel volume+chamber volume+dead volume+estimated evaporation volume). Furthermore, this will also help determine how much liquid is dispensed into the cartridge and if it is sufficient for the assay.

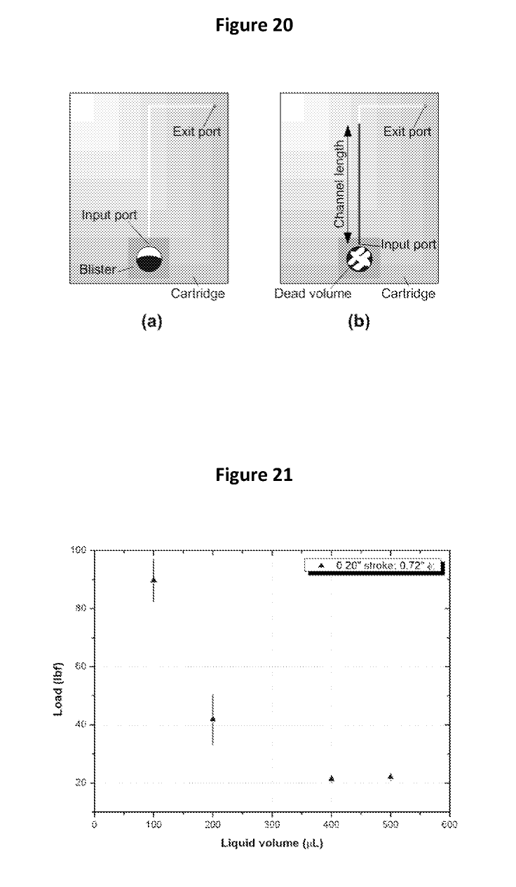

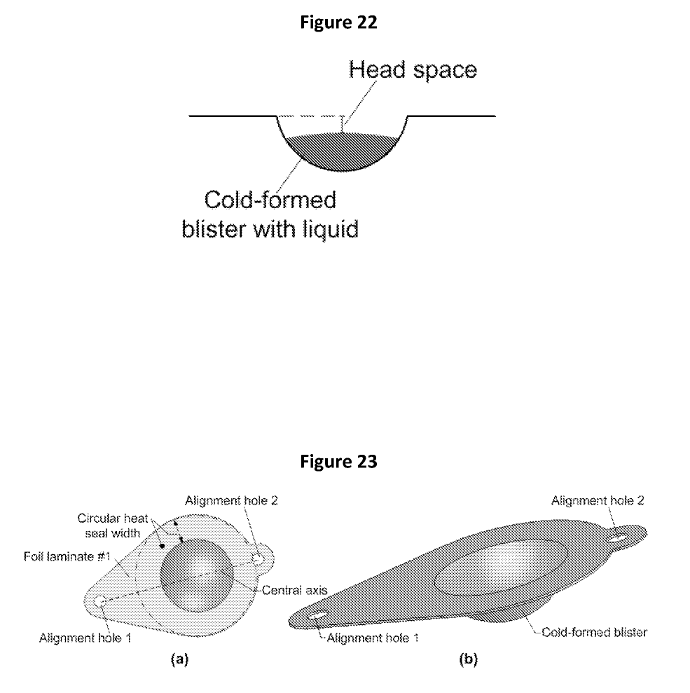

To determine the dead volume, various blisters were crushed on a characterization cartridge. The liquid was dispensed into one long channel that was previously calibrated to correlate length with liquid volume. Therefore, the dead volume can be defined as below. See FIG. 20 for a sketch of the cartridge and concept of determining the dead volume.

Dead volume=Total start volume in blister−volume dispensed into channel

The following types of blisters were tested:
0.55″ diameter
   0.150″ stroke
   150 μL
0.72″ diameter
   0.135″, 0.15″, 0.18″, and 0.20″ stroke
   300, 350, 400, 500, 550, 575, and 600 μL

The raw data is shown in

Table 1. The ‘total volume fill capacity’ value is adapted from FIG. 19. The table shows the average dispensed volume, as well as the respective dead volume and its ratio to both the total volume fill capacity and actual dispensed liquid volume. It indicates that a larger dead volume ensues for smaller blisters, and also provides information on the minimum liquid volume that should be stored in the blister (i.e., equal to the dead volume). It also shows that for the liquid volumes and strokes tested, the dead volume is relatively constant.

TABLE 1
Raw data showing the dispensed volume for each blister type, and its
respective dead volume (left behind in the blister). A ratio of the dead volume to both the
total liquid volume fill capacity and actual dispensed volume is also reported here.

| Blister type | Elution | Oil | | | Lysis | | | |
|---|---|---|---|---|---|---|---|---|
| Stroke (in) | 0.15 | 0.135 | 0.15 | 0.18 | 0.20 | | | |
| Dispensed vol (μL) | 150 | 300 | 350 | 400 | 500 | 550 | 575 | 600 |
| Total volume fill capacity | 237 | 519 | 567 | 692 | 823 | 823 | 823 | 823 |
| (μL) | | | | | | | | |
| Ave vol dispensed (μL) | 109.3 | 215.1 | 245.4 | 310.2 | 392.0 | 423.3 | 459.3 | 484.1 |
| S.D. vol dispensed (μL) | 11.2 | 29.4 | 25.7 | 13.2 | 27.0 | 22.3 | 20.0 | 12.7 |
| Ave dead vol (μL) | 40.8 | 84.9 | 104.6 | 89.8 | 108.0 | 126.7 | 115.7 | 115.9 |
| Dead vol: Total vol fill | 0.17 | 0.16 | 0.18 | 0.13 | 0.13 | 0.15 | 0.14 | 0.14 |
| capacity | | | | | | | | |
| Dead vol: Dispensed | 0.27 | 0.28 | 0.30 | 0.22 | 0.22 | 0.23 | 0.20 | 0.19 |
| blister vol | | | | | | | | |

### Example 5

### Liquid Volume Vs. Force

With varying liquid volumes inside a given blister, its effect on the force required to burst it open will also change. This is due to the amount of air that is present in the blister. While air can be compressed, liquids cannot, and with higher volumes of air, the more force will be required to burst the peelable heat seals. The amount of air in the blister is preferable reduced as much as possible: (1) during transport at lower ATA (atmospheres absolute) where perhaps, the cabin is not pressurized sufficiently, an increased amount of air in a blister can began to expand and begin to peel the heat seal; (2) reducing the amount of air will help realize consistent and uniform forces to burst open the blisters (i.e., more air means higher standard deviation in forces).

Experiments have been done to date to determine how liquid volumes for a given blister geometry affects the force. Here, the following parameters were tested.

0.72″ diameter blister

0.20″ stroke

100, 200, 400, and 500 μL liquid volume (this corresponds to 12, 24, 49, and 61% total volume fill capacity—823 μL)

The resulting data is shown in FIG. 21. The data plot shows that the force increases at low liquid volumes because of the higher volume of air. Furthermore, the standard deviation is significantly larger for low volumes, which indicates high variability from blister to blister. This is undesirable in actual experiments. When liquid volumes are 400 μL or larger, the force is reduced to more workable values, and the variability across blisters all but disappears.

### Example 6

### Additional Designs

This example describes additional blister pack designs. However, since the original submission, we have realized issues with this design. In some embodiments, small channels are created during the bonding process between foil laminate and the transfer adhesive (See e.g., FIG. 8). These channels are created due to the step difference between the transfer adhesive and foil laminate (caused by the thickness of the lidstock foil laminate). The creation of these channels cause an increase in the dead volume when a blister is burst since liquid can wick into these locations. It places high demand on ensuring the bonding technique minimizes this channeling. In some embodiments, a modified blister packaging design that minimizes dead volume is utilized.

Once a blister has been cold formed, two alignment holes are punched through the blister foil laminate, as shown in FIG. 23. Here, the alignment holes, which pass through the central axis point of the blister, are punched after the cold forming. However, it is feasible to perform this operation simultaneously with the cold forming operation. The alignment holes are positioned such that they are outside the circular heat seal perimeter (shown in FIG. 23(a)). The holes serve to align the blister during the heat sealing process (i.e., align foil laminate #1 with foil laminate #2) and manufacturing/assembly of the overall disposable (e.g., integrating and positioning the packaged reagent blister with the rigid test cartridge).

Heat sealing occurs between foil laminate #1 and foil laminate #2 (designated as ‘lidstock’). Three holes are punched into the lidstock prior to heat sealing. See FIG. 2424. Just as with the foil laminate #1, two holes are punched for alignment (both for heat sealing subsequent integration with the rigid test cartridge). The third hole is a liquid port hole which serves as the exit port for the liquid when the blister is crushed. It is positioned just outside the circular heat seal perimeter.

The cold-formed blister and lidstock are prepared for the heat sealing process, which is briefly outlined in FIG. 25. Retractable pins are used to position and align the cold-formed blister with the lidstock via the punched alignment holes. This method can subsequently be used as registration marks (alignment) during manufacturing.

The heat seal band used for this application is a donut-shaped heat seal band with extensions on the side, as shown in FIG. 2626(a). This facilitates a vapor and liquid tight heat seal bond, overlapping with the three punched holes.

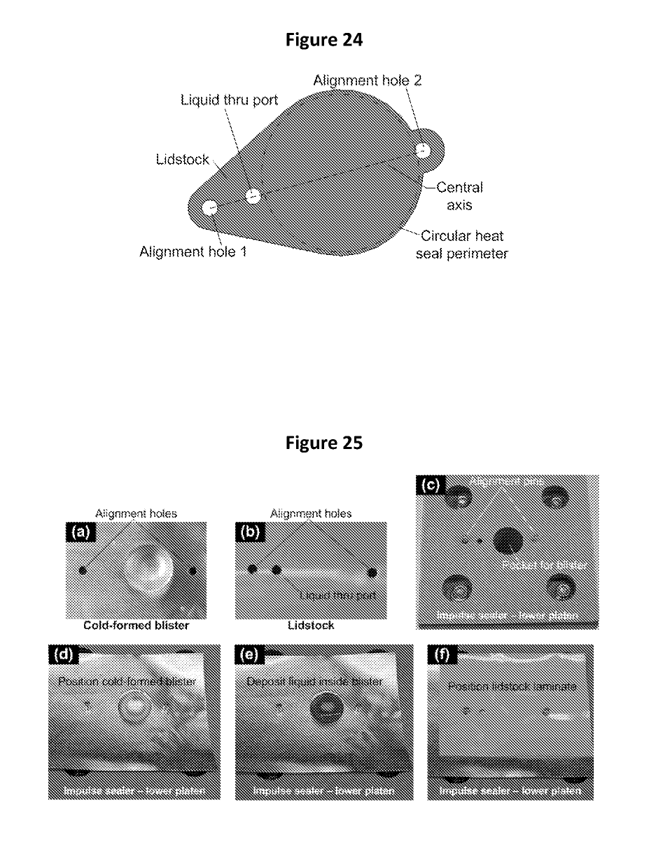

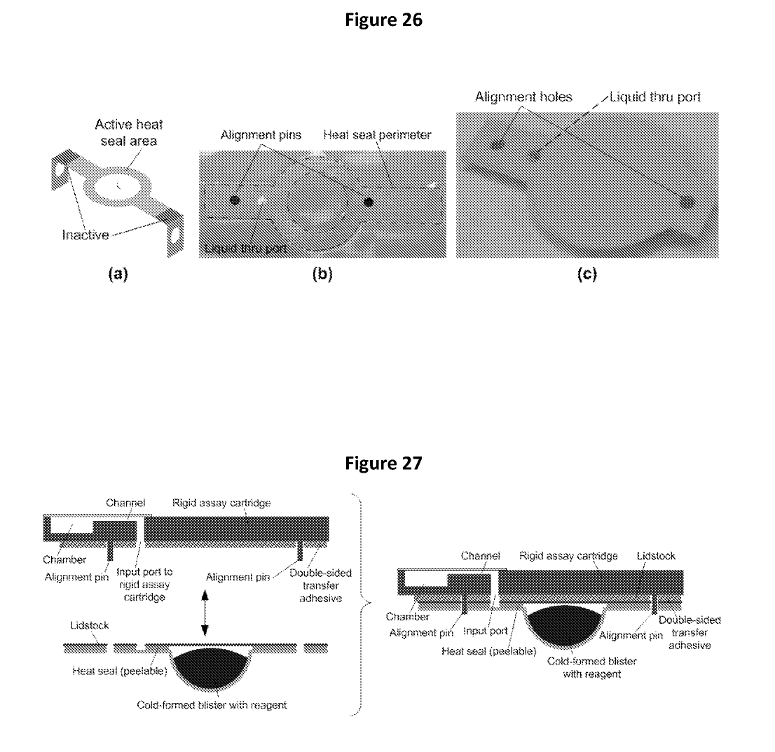

While only one blister is demonstrated in FIGS. 23-26, this method can be extended to the design and manufacturing of multiple blisters where, for example, more than one blister is required for a given test cartridge assay.

Furthermore, this overall design facilitates easy attachment to the test cartridge (e.g., using transfer adhesive) since during the adhesive bonding, the blister does not experience any variation in height. The liquid thru port punch hole and design/geometry of the heat seal band facilitates smooth bonding of the lidstock to the cartridge with no chance of liquid leakages or channeling. See FIG. 27.

Since both the foil laminate #1 and lidstock extend equally across the packaged blister, there is no step difference when bonding the blister to the rigid assay cartridge (via the double-sided transfer adhesive), preventing the creation of any channels.

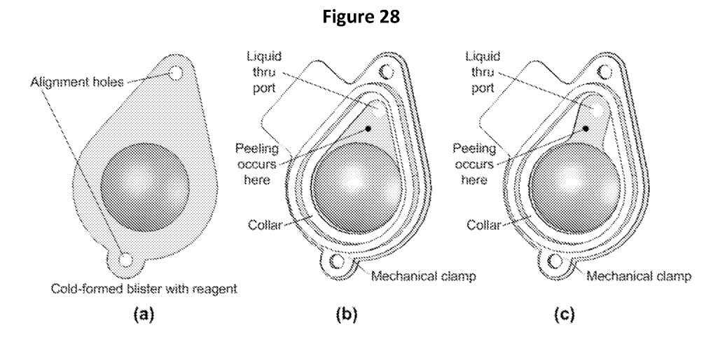

The purpose of the mechanical clamp in bursting blisters are: (1) to assist in a directional heat seal peeling process to burst the blister and deliver the liquid, (2) to provide uniform pressure along the circular heat seal perimeter at the edge of the cold-formed blister (minimize the gap between the edge of a cold-formed blister and mechanical clamp collar) such that the peeling only occurs in the direction towards the liquid thru port, and (3) to guide a mechanical plunger which applies force on the blister and eventually peels it open. See FIG. 28. This minimizes the liquid dead volume (e.g., liquid volume left behind in a completely crushed blister) and further ensures that the blister heat seal peels immediately in the direction of interest. If the gap is not minimized, it can cause the peeling can start occurring in a random direction which increases the liquid volume left behind, reducing the actual volume available for the rigid test cartridge, and/or when actively monitoring the forces required to crush and peel open a blister, it may take multiple instances to successfully direct the peeling towards the liquid thru port.

All publications, patents, patent applications and sequences identified by accession numbers mentioned in the above specification are herein incorporated by reference in their entirety. Although the invention has been described in connection with specific embodiments, it should be understood that the invention as claimed should not be unduly limited to such specific embodiments. Modifications and variations of the described compositions and methods of the invention that do not significantly change the functional features of the compositions and methods described herein are intended to be within the scope of the following claims.

1. An assay system, comprising:
a) a liquid packaging component comprising one or more liquid storage compartments, wherein each of said liquid storage compartments comprises a liquid and is a burstable seal:
b) a seal bursting component configured to burst said burstable seal; and
c) an assay device configured to accept liquid from said one or more liquid storage compartments.

2. The system of claim 1, wherein said burstable seal is a foil laminate.

3. The system of claim 2, wherein said foil is aluminum foil.

4. The system of claim 1, wherein said seal bursting component comprises a plunger that compresses said one or more liquid storage compartments under conditions such that said burstable seal is peeled open.

5. The system of claim 4, wherein said plunger is driven manually or by one or more motors.

6. (canceled)

7. The system of claim 1, wherein said liquid packaging component is connected by a fluid conduit to a chamber within said assay device.

8. The system of claim 1, wherein said liquid packaging component and a chamber within said assay device are in direct contact.

9. (canceled)

10. The system of claim 1, wherein said one or more liquid storage compartments each comprise less than 50% air by volume.

11. (canceled)

12. The system of claim 1, wherein said liquid storage compartment comprise a mechanical clamp that applies uniform pressure across a portion of the burstable seal.

13. The system of claim 12, wherein said clamp does not apply pressure to the portion of said burstable seal that is intended for communication with said assay device.

14. The system of claim 1, wherein said one or more liquid storage compartments comprise reagents for performing an assay.

15. (canceled)

16. The system of claim 1, wherein said liquid packaging component further comprises one or more alignment pins to secure said liquid storage compartment to said liquid packaging component.

17. An assay method, comprising:
contacting a liquid packaging component comprising one or more liquid storage compartments with a seal bursting component, wherein each of said one or more liquid storage compartments comprises a liquid and a burstable seal, and wherein the seal bursting component is configured to burst said burstable seal under conditions such that said liquid is transported to an assay device configured to accept liquid from said one or more liquid storage compartments.

18. The method of claim 17, wherein said burstable seal is a foil laminate.

19. The method of claim 18, wherein said foil is aluminum foil.

20. The method of claim 17, wherein said seal bursting component comprises a plunger that compresses said one or more liquid storage compartments under conditions such that said burstable seal is peeled open.

21. The method of claim 20, wherein said plunger is driven manually or by one or more motors.

22. (canceled)

23. The method of claim 17, wherein said liquid packaging component is connected by a fluid conduit to a chamber within said assay device.

24. The method of claim 17, wherein said liquid packaging component and a chamber within said assay device are in direct contact.

25. (canceled)

26. (canceled)

27. The method of claim 17, wherein said one or more liquid storage compartments each comprise less than 60% air by volume.

28. (canceled)

29. The method of claim 17, wherein said one or more liquid storage compartments comprise a mechanical clamp that applies uniform pressure across a portion of the burstable seal.

30. The method of claim 29, wherein said clamp does not apply pressure to a portion of said burstable seal that is in fluid communication with said assay device.

31. The method of claim 17, wherein said one or more liquid storage compartments comprise a reagent for performing an assay.

32. The method of claim 17, wherein said assay is selected from the group consisting of a diagnostic assay and a research assay.

33. The method of claim 17, wherein said liquid packaging component further comprises one or more alignment pins to secure said one or more liquid storage compartments to said liquid packaging component.

34. The system of claim 1, wherein the one or more liquid storage compartments comprise one or more holes for alignment or liquid delivery.

35. The system of claim 1, wherein the liquid packaging component comprises a liquid storage compartment comprising an elution buffer or a lysis buffer and a liquid storage compartment comprising an oil.

36. The system of claim 1, wherein the liquid packaging component comprises a liquid storage compartment comprised of a laminate film shaped to form a hemispherical blister with a perimeter and a lidstock material sealed to the laminate at the perimeter.

37. The system of claim 36, wherein the liquid packaging component further comprises a clamp positioned around the blister.

38. The method of claim 17, wherein the one or more liquid storage compartments comprise one or more holes for alignment or liquid delivery.

39. The method of claim 17, wherein the liquid packaging component comprises a liquid storage compartment comprising an elution buffer or a lysis buffer and a liquid storage compartment comprising an oil.

40. The method of claim 17, wherein the liquid packaging component comprises a liquid storage compartment comprised of a laminate film shaped to form a hemispherical blister with a perimeter and a lidstock material sealed to the laminate at the perimeter.

41. The method of claim 40, wherein the liquid packaging component further comprises a clamp positioned around the blister.
